(19) United States
(12) Patent Application Publication
(10) Pub. No.: US 2018/0207081 A1
(43) Pub. Date: Jul. 26, 2018

(54) GEL-FORM COMPOSITION

(71) Applicant: SUMITOMO SEIKA CHEMICALS CO., LTD., Hyogo (JP)

(72) Inventors: Akio NAKATSUKA, Himeji-shi (JP); Makiko KAWANO, Himeji-shi (JP); Yousuke NAKAMURA, Himeji-shi (JP)

(73) Assignee: SUMITOMO SEIKA CHEMICALS CO., LTD., Hyogo (JP)

(21) Appl. No.: 15/748,031

(22) PCT Filed: Aug. 1, 2016
(86) PCT No.: PCT/JP2016/072469

§ 371 Date: Jan. 26, 2018

(30) Foreign Application Priority Data
Aug. 4, 2015 (JP) 2015-154193

(51) Int. Cl. A61K 8/81; A61K 8/34; A61Q 19/00

(52) U.S. Cl. CPC A61K 8/8147; A61K 2800/48; A61Q 19/00; A61K 8/345

(57) ABSTRACT

Provided is a gelled composition that exhibits, despite having 50 mass % or more of a polyhydric alcohol content, a strong thickening effect, while providing a smooth feeling of use on the skin, and even excellent dispersibility. The gelled composition of the present invention contains (A) a polymer of an ethylenically unsaturated carboxylic acid monomer crosslinked with a water-soluble crosslinking agent, (B) a polyhydric alcohol, and (C) water, wherein component (A) is present in an amount of 0.1 to 5 mass %, component (B) is present in an amount of 50 to 95 mass %, and component (C) is present in an amount of 1 to 10 mass %.

### TECHNICAL FIELD

The present invention relates to a gelled composition, more specifically to a gelled composition for use in warm gels, cosmetic scrubs, sheet masks, nourishing cosmetic materials, etc.

### BACKGROUND ART

Gelled materials have a wide range of applications, such as warm gels, scrub cosmetics, sheet masks, and nourishing cosmetic materials. Gelled materials are gelled and develop the function of imparting a thickening effect to various materials because they contain a thickening agent. For this thickening agent, hydrophilic thickening agents are widely used. Specific examples include natural thickening agents, such as xanthan gum and guar gum; semisynthetic thickening agents, such as hydroxyethyl cellulose and carboxy methylcellulose; and synthetic thickening agents, such as carboxyvinyl polymers and polyethylene oxide. Of these thickening agents, carboxyvinyl polymers are heavily used due to their cheap price, strong thickening effect, and ability to form a gel in small amounts.

A gelled material contains not only the thickening agent but also liquid components, such as alcohols and water. Because changes in the proportion of these components lead to different physical properties or different costs for the gelled material, it is important to adjust the proportion of each component of the material. For example, application of a gelled material in a cosmetic material, such as a warm gel, requires that the gelled material contain 50 mass % or more of a polyhydric alcohol; and a variety of thickening agents are proposed (see, for example, Patent Literature 1 to 3). A gelled material with a higher content of a polyhydric alcohol has an advantage in making effective use of heat from mixing water with the polyhydric alcohol, and is particularly suitable for use in cosmetic materials, such as warm gels.

### CITATION LIST

### Patent Literature

Patent Literature 1: JP2010-37272A
Patent Literature 2: JP2010-265180A
Patent Literature 3: JP2003-300826A

### SUMMARY OF INVENTION

### Technical Problem

The technique disclosed in Patent Literature 1 is, however, unlikely to provide a desired thickening effect, because increasing the polyhydric alcohol content and decreasing the water content of a gelled material that contains a carboxyvinyl polymer as a thickening agent makes it difficult to increase the viscosity. The technique disclosed in Patent Literature 2 uses a polyethylene glycol fatty acid ester; however, the polyethylene glycol fatty acid ester is unlikely to provide a smooth feel on the skin, and the use thereof as a cosmetic material is more likely to be avoided. Patent Literature 3 also discloses a warm cosmetic material containing a starch-sodium acrylate graft polymer as a thickening agent, but it is difficult to provide a smooth feeling of use on the skin by this cosmetic material as well. Additionally, the poor dispersibility of the thickening agent in a polyhydric alcohol makes it difficult to prepare warm cosmetic materials.

The present invention solves these problems, and an object of the invention is to provide a gelled composition that exhibits, despite having 50 mass % or more of a polyhydric alcohol content, a strong thickening effect, while providing a smooth feeling of use on the skin, and even excellent dispersibility.

### Solution to Problem

The present inventors conducted extensive research, and found that the use of a specific polymer as a thickening agent can achieve the object, and completed the present invention.

Specifically, the present invention is as shown below.

### Item 1.

A gelled composition comprising the following components (A) to (C):

(A) a polymer of an ethylenically unsaturated carboxylic acid monomer crosslinked with a water-soluble crosslinking agent;
(B) a polyhydric alcohol; and
(C) water,
wherein component (A) is present in an amount of 0.1 to 5 mass %, component (B) is present in an amount of 50 to 95 mass %, and component (C) is present in an amount of 1 to 10 mass %, based on the total amount of the gelled composition.

### Item 2.

The gelled composition according to Item 1, wherein the polymer has a mean particle size of 5 to 40 μm.

### Item 3.

The gelled composition according to Item 1 or 2, wherein the water-soluble crosslinking agent contains at least one compound selected from the group consisting of N,N′-methylenebis acrylamide, ethylene glycol dimethacrylate, ethylene glycol diglycidyl ether, polyethylene glycol dimethacrylate, polyethylene glycol diglycidyl ether, and sucrose allyl ether.

### Item 4.

The gelled composition according to any one of Items 1 to 3, wherein the water-soluble crosslinking agent contains sucrose allyl ether, and the degree of etherification of the sucrose allyl ether is 2.0 to 3.5.

### Item 5.

A cosmetic material comprising the gelled composition according to any one of Items 1 to 4.

### Item 6.

The cosmetic material according to Item 5, which is a warm gel cosmetic material.

### Advantageous Effects of Invention

Because of the specific polymer contained as an essential component, the gelled composition of the present invention exhibits, despite having 50 mass % or more of the polyhydric alcohol content, a strong thickening effect and provides a smooth feeling of use on the skin. In addition, the gelled composition exhibits excellent dispersibility of the polymer as a thickening agent in the polyhydric alcohol.

### BRIEF DESCRIPTION OF DRAWINGS

FIG. 1 is a photograph that illustrates a thickening agent dispersed in a dispersion medium. FIG. 1(a) illustrates the dispersion state of the polymer used in Example 1, and FIG. 1(b) illustrates the dispersion state of the polymer used in Comparative Example 1.

### DESCRIPTION OF EMBODIMENTS

The gelled composition of the present invention comprises the following components (A) to (C):

(A) a polymer of an ethylenically unsaturated carboxylic acid monomer crosslinked with a water-soluble crosslinking agent;
(B) a polyhydric alcohol; and
(C) water,
wherein component (A) is present in an amount of 0.1 to 5 mass %, component (B) is present in an amount of 50 to 95 mass %, and component (C) is present in an amount of 1 to 10 mass %, based on the total amount of the gelled composition.

In the gelled composition, the polymer of an ethylenically unsaturated carboxylic acid monomer crosslinked with a water-soluble crosslinking agent (A) acts as a thickening agent. The polyhydric alcohol (B) and water (C) are a dispersion medium for the polymer (A).

The polymer (A) is a polymer of an ethylenically unsaturated carboxylic acid monomer, and is crosslinked with a water-soluble crosslinking agent. Specifically, the polymer (A) contains repeating structural units derived from an ethylenically unsaturated carboxylic acid monomer. The water-soluble crosslinking agent cross-links the polymer chain formed of repeating structural units derived from an ethylenically unsaturated carboxylic acid monomer.

Examples of the ethylenically unsaturated carboxylic acid monomer include (meth)acrylic acid, 2-(meth)acrylamide-2-methylpropanesulfonic acid, and alkali metal salts thereof; nonionic monomers, such as (meth)acrylamide, N,N-dimethylacrylamide, 2-hydroxyethyl (meth)acrylate, and N-methylol (meth)acrylamide; and amino-containing unsaturated monomers, such as diethylaminoethyl (meth)acrylate, and diethylamino propyl(meth)acrylate, and quaternary products thereof. Alkali metals of alkali metal salts include lithium, sodium, and potassium. Of these, (meth)acrylic acid, alkali metal salts thereof, acrylamide, methacrylamide, and N,N-dimethylacrylamide are preferable. These ethylenically unsaturated carboxylic acid monomers may be used singly or in a combination of two or more.

In this specification, “(meth)acrylic” means “acrylic or methacrylic.” Specifically, “(meth)acrylic acid,” for example, is synonymous with “acrylic acid or methacrylic acid.”

Preferable water-soluble crosslinking agents include compounds having 2 or more reactive functional groups and/or polymerizable unsaturated groups. Specific examples include a glycidyl group and an isocyanate group. Compounds having 2 or more glycidyl groups include ethylene glycol diglycidyl ether and polyethylene glycol diglycidyl ether. Compounds having 2 or more polymerizable unsaturated groups include N,N′-methylenebis acrylamide, ethylene glycol dimethacrylate, polyethylene glycol dimethacrylate, and sucrose allyl ether. These water-soluble crosslinking agents may be used singly or in a combination of two or more. Of these, sucrose allyl ether is preferable for use. The use of sucrose allyl ether particularly makes it easy to disperse the obtained polymer (A) in a dispersion medium, and further improves the smooth feeling of use on the skin.

When the water-soluble crosslinking agent is sucrose allyl ether, the degree of etherification is not particularly limited. For example, the degree of etherification can be 2.0 to 3.5. The degree of etherification within this range facilitates the crosslinking reaction due to the presence of sufficient functional groups involved in the crosslinking reaction (i.e., allyl groups). The degree of etherification within this range also makes the solubility in water unlikely to decrease, thereby facilitating the crosslinking reaction of the sucrose allyl ether with the ethylenically unsaturated carboxylic acid monomer in an aqueous phase. A more preferable degree of etherification is 2.0 to 3.0.

As used here, the degree of etherification is defined as the average of molar ratios of allyl ether groups to sucrose. Specifically, hydroxyl groups remaining in sucrose allyl ether are reacted with acetic anhydride in pyridine, and the degree of etherification is calculated from the amount of acetic anhydride consumed during this reaction.

Sucrose allyl ether can be produced by a known method. For example, allyl ether can be produced by a method in which sodium hydroxide is added to a sucrose aqueous solution to convert sucrose into alkaline sucrose, and allyl bromide is added thereto to perform etherification. Sucrose allyl ether can be efficiently obtained by adjusting, at this stage, the amount of allyl bromide relative to the amount of sucrose so as to fall within the range of preferably 2 to 6-fold moles, and more preferably 2 to 5-fold moles. The reaction temperature for etherification can be, for example, about 80° C. Typically, after addition of allyl bromide, the reaction ends in about 3 hours. An alcohol is added to the aqueous phase separated from the reaction mixture, and precipitated salts are filtered, followed by distilling off extra alcohol and water, thereby obtaining sucrose allyl ether.

The polymer (A) can be produced by a known method. Examples include a method including the step of polymerizing an ethylenically unsaturated carboxylic acid monomer in the presence of a water-soluble crosslinking agent by a suspension polymerization method. Among suspension polymerization methods, preferable is an inverse suspension polymerization method in which polymerization reaction is performed by dispersing droplets of an aqueous phase containing an ethylenically unsaturated carboxylic acid monomer, a water-soluble crosslinking agent, and water in a hydrophobic solvent. Because almost all particles of the polymer obtained by this inverse suspension polymerization method have a spherical shape, a smooth feeling of use on the skin is likely to increase, and their dispersibility in a dispersion medium is also likely to increase. Thus, such a polymer is particularly preferable for use in the gelled composition of the present invention.

The proportions of the ethylenically unsaturated carboxylic acid monomer and the water-soluble crosslinking agent are not particularly limited. For example, the amount of the water-soluble crosslinking agent relative to the amount of the ethylenically unsaturated carboxylic acid monomer can be 0.01 to 2.0 mass %. The proportions in this range can enhance particularly the thickening effect of the gelled composition and make it easier for the polymer (A) to disperse in a dispersion medium, thus increasing the smooth feeling of use on the skin. The amount of the water-soluble crosslinking agent relative to the amount of the ethylenically unsaturated carboxylic acid monomer is more preferably 0.05 to 1.0 mass %.

Examples of the hydrophobic solvent include petroleum-based hydrocarbon solvents selected from aliphatic hydrocarbons, alicyclic hydrocarbons, and aromatic hydrocarbons. Aliphatic hydrocarbons include n-pentane, n-hexane, and n-heptane. Alicyclic hydrocarbons include cyclopentane, methylcyclopentane, cyclohexane, and methylcyclohexane. Aromatic hydrocarbons include benzene, toluene, and xylene. In particular, at least one hydrophobic solvent selected from n-hexane, n-heptane, cyclohexane, or toluene can suitably be used as an industrially versatile solvent. The amount of the hydrophobic solvent can be, for example, 100 to 200 parts by mass, per 100 parts by mass of the aqueous phase containing the ethylenically unsaturated carboxylic acid monomer, etc.

The aqueous phase containing the ethylenically unsaturated carboxylic acid monomer, etc., or the hydrophobic solvent may contain other components, such as a surfactant and a radical initiator.

The surfactant is used largely for the purpose of stabilizing the suspension state during polymerization. The surfactant is not particularly limited, as long as the surfactant is typically used in inverse suspension polymerization. Preferable surfactants for use include sorbitan fatty acid esters, polyglycerin fatty acid esters, sucrose fatty acid esters, sorbitol fatty acid esters, modified polyethylene wax, modified polypropylene wax, polyvinyl alcohols, polyethylene oxide, cellulose ethers (e.g., hydroxyethyl cellulose and ethyl cellulose), sodium alkylbenzene sulfonate, and polyoxyethylene alkylphenyl ether sulfate. These can be used singly, or in a combination of two or more.

The amount of the surfactant is preferably 0.1 to 5.0 mass %, and more preferably 0.2 to 3.0 mass %, relative to the ethylenically unsaturated carboxylic acid monomer.

The radical initiator is not particularly limited, as long as the radical initiator is typically used in radical polymerization. Preferable radical initiators for use include potassium persulfate, ammonium persulfate, sodium persulfate, and azo-based initiators. For example, 2,2′-azobis(2-methylpropionamidine) dihydrochloride can be used as a radical initiator.

The amount of the radical initiator is preferably 0.01 to 0.5 mass %, and more preferably 0.02 to 0.2 mass %, relative to the ethylenically unsaturated carboxylic acid monomer.

In inverse suspension polymerization, the size of a droplet containing the ethylenically unsaturated carboxylic acid monomer, etc., is closely related to the particle size of the polymer obtained. Although it depends on the conditions, such as the reactor and production scale, it is preferable to perform inverse suspension polymerization at a stirring rate of 600 to 1000 rotations/min, for example, if using a 2000-mL flask as a reactor; this provides a polymer having a particle size and a shape suitable for use in the gelled composition of the present invention. The molecular weight and the degree of crosslinkage of the crosslinked polymer can be adjusted by the amount of the water-soluble crosslinking agent added.

Other conditions for polymerization reaction, such as the reaction temperature and reaction time, can also suitably be adjusted. The reaction temperature is, for example, 50 to 80° C., and the reaction time is, for example, 30 minutes to 3 hours. When a 2000-mL flask is used as a reactor, the bath temperature can be adjusted to 60° C. to start polymerization reaction. In this case, the start of polymerization reaction can be confirmed by an elevation of the temperature inside the reactor to about 70° C. due to heat of polymerization. Thereafter, the polymerization reaction continues for about 30 minutes to 3 hours and then typically ends. After completion of the reaction, the bath temperature is increased to distill off water and the petroleum-based hydrocarbon solvent inside the reactor, thereby obtaining a polymer.

The mean particle size of the polymer (A) is preferably 5 to 40 μm, more preferably 5 to 30 μm, and particularly preferably 5 to 25 μm. The mean particle size refers to the value as determined by dispersing the polymer (A) in n-hexane and measuring the dispersed polymer with a laser diffraction particle size distribution analyzer. The analyzer for use may be an SALD2000 (manufactured by Shimadzu Corporation). A mean particle size of 5 μm or more can avoid the difficulty in handling fine powder, and a mean particle size of 40 μm or less can make it easy for the gelled composition to provide a smooth feeling of use on the skin.

The shape of the polymer (A) is not particularly limited, and may be truly spherical or elliptical as long as the polymer (A) is in the form of particles.

The polymer (A) contains repeating structural units derived from an ethylenically unsaturated carboxylic acid monomer, and carboxyl groups in these repeating structural units may be neutralized to form salts (e.g., metal salts or ammonium salts). In this case, not all carboxyl groups need to be neutralized, and part of the carboxyl groups may be neutralized. Specifically, the polymer may be a partially neutralized product. Such polymers include crosslinked products of polyacrylates.

When the polymer (A) is a partially neutralized product, 40 to 70 mol % of all the repeating structural units derived from the ethylenically unsaturated carboxylic acid monomer may be neutralized. When the polymer (A) is a partially neutralized product, and the degree of neutralization is, for example, about 40 to 70 mol %, the pH can be easily adjusted to about 6 to 7, which can typically be applied to cosmetics.

The partially neutralized product can be obtained by treating the polymer with an alkali metal salt, such as sodium hydroxide. Alternatively, the partially neutralized product can be obtained by neutralizing the ethylenically unsaturated carboxylic acid monomer beforehand and polymerizing this monomer.

The polyhydric alcohol (B) includes, but is not limited to, glycerin, 1,3-butanediol (butylene glycol), propylene glycol, dipropylene glycol, diglycerin, isoprene glycol, and 1,2-pentanediol. These polyhydric alcohols can be used singly, or in a combination of two or more.

The type of water (C) is not particularly limited, and examples include a variety of water, such as pure water, distilled water, ion-exchanged water, purified water, and tap water. The pH of the water is also not limited, and the pH can be, for example, about 6 to 7.

The gelled composition of the present invention contains 0.1 to 5 mass % of the polymer (A), 50 to 95 mass % of the polyhydric alcohol (B), and 1 to 10 mass % of the water (C), based on the total amount of the gelled composition. The amount of the polymer (A) is on a dry mass basis.

Less than 0.1 mass % of the polymer (A) based on the total amount of the gelled composition results in unsatisfactory smoothness on the skin, failing to provide an excellent feeling of use. More than 5 mass % of the polymer (A) also results in an overly hard gelled composition, deteriorating the feeling of use. The amount of the polymer (A) is preferably 0.1 to 2 mass %, and more preferably 0.2 to 1 mass %.

Less than 50 mass % of the polyhydric alcohol (B) based on the total amount of the gelled composition fails to provide a warming effect in the use of the gelled composition as a warm gel cosmetic material. More than 95 mass % of the polyhydric alcohol (B) leads to an insufficient water content for thickening, and makes it unlikely to obtain a warming effect. The amount of the polyhydric alcohol (B) is preferably 50 to 95 mass %, and more preferably 55 to 95 mass %.

Less than 1 mass % of the water (C) based on the total amount of the gelled composition leads to an insufficient water content for thickening. More than 10 mass % of the water (C) hinders the warming effect due to excess water. The amount of the water (C) is preferably 2 to 7 mass %.

The gelled composition of the present invention may contain other additives, for example, warming effect-imparting components, such as sodium polyacrylate, phenoxyethanol, Capsicum extracts, and zeolite; oil components, such as olive oil; plant extracts; and antimicrobial agents, as long as effects of the invention are not impaired. When the gelled composition contains other additives, the amount of other additives is preferably 35 mass % or less based on the total amount of the gelled composition.

The gelled composition of the present invention can be prepared, for example, by adding predetermined amounts of the polymer (A), the polyhydric alcohol (B), and the water (C), optionally with other additives, and mixing them. Examples of the method for producing the gelled composition include a method including the following steps sequentially: the step of dispersing the polymer (A) in the polyhydric alcohol (B), the step of adding a predetermined amount of water (C) thereto to thicken the dispersion, and the step of mixing the remaining polyhydric alcohol (B) with the dispersion. In particular, the polymer (A) is excellent in dispersibility in a dispersion medium, such as the polyhydric alcohol (B), thus making it easy to prepare the gelled composition.

The gelled composition of the present invention has a range of applications. In particular, despite having 50 mass % or more of the polyhydric alcohol content, the gelled composition of the present invention can exhibit a strong thickening effect and provide a smooth feeling of use on the skin, while even exhibiting excellent dispersibility. Thus, the gelled composition is suitable for use in cosmetic materials, for example, skin-care cosmetics, body-care cosmetics, and fragrance cosmetics. In particular, the high polyhydric alcohol content enables effective use of heat from mixing water with the polyhydric alcohol, thus making the gelled composition suitable for cosmetic materials, such as warm gels. Moreover, the gelled composition can be used in massage gels, sheet masks, cosmetic scrubs, nourishing cosmetic materials, and like materials.

### EXAMPLES

The following describes the present invention with reference to Examples in more detail. However, the present invention is not limited to these Examples.

### Synthesis of Water-Soluble Crosslinking Agent

### Production Example 1

A stirrer, a reflux condenser, and a dropping funnel were attached to a 1000-mL separable flask. In this flask, 48 g of sodium hydroxide (1.2 moles) was dissolved in 144 g of water. Subsequently, 136.8 g of sucrose (0.4 moles) was added thereto, and the mixture was stirred at 70 to 85° C. for 120 minutes, thereby obtaining an alkaline sucrose aqueous solution. 145.2 g of allyl bromide (1.2 moles) was added dropwise to the obtained alkaline sucrose aqueous solution at 70 to 85° C. over the course of 1.5 hours, and then the mixture was reacted at 80° C. for 3 hours to allyl-etherify the sucrose. After cooling, 440 g of water was added thereto, and excessive oil was removed with a separatory funnel, thereby obtaining a crude sucrose allyl ether aqueous solution. Hydrochloric acid was added to the obtained crude sucrose allyl ether aqueous solution to adjust the pH to 6 to 8, and water was removed with a rotary evaporator until the mass of the aqueous solution reached 480 g. Subsequently, 200 g of ethanol was added thereto, and salts, such as sodium bromide, (by-products) were precipitated, followed by removing the precipitates from the aqueous solution through filtration. Further, excessive water was removed from the aqueous solution with an evaporator, thereby obtaining 166 g of sucrose allyl ether with a degree of etherification of 2.4.

Synthesis of Polymer of Ethylenically Unsaturated Carboxylic Acid Monomer Crosslinked with Water-Soluble Crosslinking Agent

### Production Example 2

A stirrer, a reflux condenser, and a dropping funnel were attached to a 500-mL separable flask. 72 g of acrylic acid and water were added to the flask to prepare 90 g of an 80 mass % acrylic acid aqueous solution. While this acrylic acid aqueous solution was cooled, 54 g of a 30 mass % sodium hydroxide aqueous solution was added dropwise thereto to neutralize the acrylic acid aqueous solution. Subsequently, 56 g of ion-exchanged water, 0.32 g of sucrose allyl ether obtained in Production Example 1 (0.35 mass % relative to the acrylic acid aqueous solution), and 0.04 g of 2,2′-azobis(2-methylpropionamidine) dihydrochloride (Wako Pure Chemical Industries, Ltd. “V-50”) were added thereto, thereby preparing an ethylenically unsaturated carboxylic acid monomer aqueous solution.

Separately, 330 g of n-heptane was added to a 2000-mL separable flask equipped with a stirrer, a reflux condenser, a dropping funnel, and a nitrogen gas inlet tube, and 2.7 g of sorbitan monostearate (NOF Corporation “NONION SP-60R”) was further added thereto, followed by dispersing and dissolving it in n-heptane. Subsequently, the previously prepared ethylenically unsaturated carboxylic acid monomer aqueous solution was added thereto. To remove the oxygen present in the atmosphere, in the starting materials in the reactor, and in the solvent, nitrogen gas was allowed to flow into the solution. While the inside of the system was replaced with nitrogen, a reaction was performed for 1 hour with the bath temperature maintained at 60° C., with stirring at a rotational frequency of 1000 rotations/minute. After completion of the reaction, water and n-heptane were distilled off, thereby obtaining 90 g of a polymer (A) formed from acrylic acid and a sodium salt thereof, which was crosslinked with sucrose allyl ether (hereinafter, the polymer obtained in Production Example 2 is referred to as “polymer A”).

### Production Example 3

The procedure of Production Example 2 was repeated, except that the stirring rotational frequency was changed to 800 rotations/minute, thereby obtaining 92 g of a polymer (B) formed from acrylic acid and a sodium salt thereof, which was crosslinked with sucrose allyl ether (hereinafter, the polymer obtained in Production Example 3 is referred to as “polymer B”).

### Production Example 4

The procedure of Production Example 2 was repeated, except that the stirring rotational frequency was changed to 600 rotations/minute, thereby obtaining 93 g of a polymer (C) formed from acrylic acid and a sodium salt thereof, which was crosslinked with sucrose allyl ether (hereinafter, the polymer obtained in Production Example 4 is referred to as “polymer C”).

### Synthesis of Carboxyvinyl Polymer

### Production Example 5

45 g of acrylic acid (0.625 moles), 0.27 g of pentaerythritol allyl ether, 150 g of n-hexane, and 0.081 g of 2,2′-azobis methyl isobutyrate (0.00035 moles) were added to a 500-mL four-necked flask equipped with a stirrer, a thermometer, a nitrogen blowing tube, and a condenser, thereby preparing a reaction mixture. The reaction mixture was stirred to homogeneously mix the starting materials, and then nitrogen gas was blown into the solution to remove oxygen present in the upper space of the reactor, in the starting materials, and in the solvent. Subsequently, the reaction mixture was reacted for 4 hours in a nitrogen atmosphere with the temperature of the reaction mixture maintained at 60 to 65° C. After completion of the reaction, the generated slurry was heated to 90° C. to distill off n-hexane, further followed by drying under reduced pressure at 110° C. at 10 mmHg for 8 hours, thereby obtaining 42 g of a carboxyvinyl polymer.

### Characteristics Evaluation of Polymers A to C and Carboxyvinyl Polymer

### Measurement of Mean Particle Size

Polymers A to C and the carboxyvinyl polymer were individually dispersed in n-hexane, and measured for their mean particle size with a laser diffraction particle size analyzer (SALD2000, a flow cell used, manufactured by Shimadzu Corporation).

### Measurement of Viscosity of 0.5 Mass % Aqueous Solution

Polymers A to C were individually mixed with water to prepare an aqueous solution with a concentration of 0.5 mass %. The carboxyvinyl polymer was adjusted to a pH of 6 with a 6.0 mass % sodium hydroxide aqueous solution as a neutralizer, and also adjusted to give a polymer concentration of 0.5 mass %. The viscosity of each 0.5 mass % aqueous solution was measured with a BH-type rotary viscometer. The rotational rate of the spindle rotor at 25° C. was set to 20 rotations per minute, and the viscosity value after 1 minute from the start of the rotation of the rotor was read. A rotor No. 6 was used for polymers A to C, and a rotor No. 7 was used for the carboxyvinyl polymer.

TABLE 1

| | | Mean | Viscosity of 0.5 Mass % |
|---|---|---|---|
| | | Particle Size | Aqueous Solution |
| | | (μm) | (mPa · s) |
| Production | Polymer A | 10 | 23,000 |
| Example 2 | | | |
| Production | Polymer B | 20 | 18,000 |
| Example 3 | | | |
| Production | Polymer C | 35 | 15,000 |
| Example 4 | | | |
| Production | Carboxyvinyl | 4 | 48,000 |
| Example 5 | Polymer | | |

Warm gels were prepared from polymers A to C and the carboxyvinyl polymer as in the following Examples and Comparative Example.

### Example 1: Warm Gel-1

The components shown in Table 2 were mixed, following the proportions indicated in Table 2, thereby preparing warm gel-1.

TABLE 2

| | |
|---|---|
| Component | Mass % |

| | | |
|---|---|---|
| 1 | Glycerin | 10.0 |
| 2 | Polymer A (Production Example 2) | 0.4 |
| 3 | Polyacrylic Acid Na | 0.002 |
| 4 | Water | 4.5 |
| 5 | Glycerin | 79.7 |
| 6 | Butylene Glycol | 5.0 |
| 7 | Phenoxy Ethanol | 0.4 |

Specifically, component 2 (corresponding to the polymer (A)) and component 3 were added to component 1 (corresponding to the polyhydric alcohol (B)), and dispersed with a jar tester at 400 rpm for 5 minutes. Component 4 (corresponding to the water (C)) was added thereto, and the mixture was stirred with a hand-held mixer for 5 minutes. Finally, a mixture solution of component 5 (corresponding to the polyhydric alcohol (B)), component 6 (corresponding to the polyhydric alcohol (B)), and component 7 was added thereto, thereby obtaining warm gel-1.

### Example 2: Warm Gel-2

The procedure of Example 1 was repeated, except that polymer A was replaced by polymer B, thereby obtaining warm gel-2.

### Example 3: Warm Gel-3

The procedure of Example 1 was repeated, except that polymer A was replaced by polymer C, thereby obtaining warm gel-3.

### Comparative Example 1: Warm Gel-4

The procedure of Example 1 was repeated, except that polymer A was replaced by the carboxyvinyl polymer, and that this carboxyvinyl polymer was neutralized with a 6.0 mass % sodium hydroxide aqueous solution, thereby obtaining warm gel-4.

### Characteristics Evaluation of Warm Gel

### Smoothness

10 testers (5 males and 5 females) evaluated the smoothness when the gels were applied, on a scale of 1 to 5 in accordance with the following criteria.

Excellent: 5 points
Good: 4 points
Ordinary: 3 points
Slightly poor: 2 points
Poor: 1 point

The average of the evaluations of the 10 testers was calculated. An average of 4 or more was rated √, and an average of 3 or more and less than 4 was rated Δ, with an average of less than 3 rated φ.

### Viscosity

The viscosity was measured with a BH-type rotary viscometer. The rotation of the rotor was started, with the rotational rate of the spindle rotor set to 20 rotations per minute at 25° C., and the viscosity value after 1 minute from the start of the rotation was read.

TABLE 3

| Examples and | | | |
|---|---|---|---|
| Comparative | | | B-type Viscosity |
| Example | Sample | Smoothness | [mPa · s] |
| Example 1 | Warm Gel-1 | 4.9 ✓ | 130,000 |
| Example 2 | Warm Gel-2 | 4.5 ✓ | 117,000 |
| Example 3 | Warm Gel-3 | 3.8 Δ | 100,000 |

| | | |
|---|---|---|
| Comparative | Warm Gel-4 | Particles clumped, a gel not formed |
| Example 1 | | |

Warm gels-1 to 3 of Examples 1 to 3 were excellent in smoothness, and the measurement results of B-type viscosity of all warm gels-1 to 3 exceeded 100,000 mPa·s, indicating that gels-1 to 3 can provide an excellent thickening effect. Comparative Example 1 for warm gel-4, however, exhibited poor dispersibility of the carboxyvinyl polymer, causing clumping, thus failing to prepare the target warm gel.

### Evaluation of Dispersibility in Glycerin

300 g of glycerin was placed in a 500-mL beaker at 25° C., and 3 g of polymer A, B, C, or the carboxyvinyl polymer was added thereto little by little, while the mixture was stirred at 400 rpm with a jar tester. The dispersibility was evaluated by visual observation.

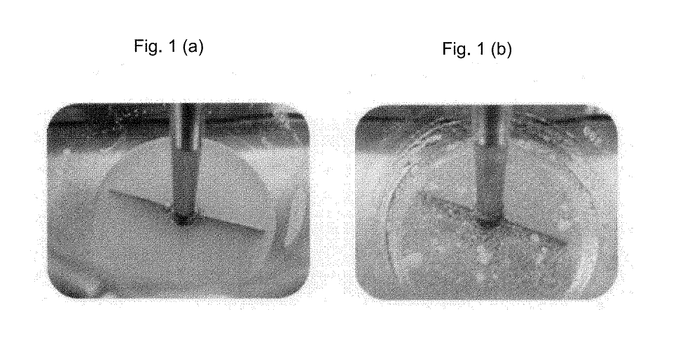

FIG. 1 shows the results of the evaluation of dispersibility in glycerin. FIG. 1(a) and FIG. 1(b) are photographs illustrating, respectively, the dispersion state of polymer A and the dispersion state of the carboxyvinyl polymer.

Polymer A of FIG. 1(a) formed an emulsion when stirred for about 5 minutes, and was homogeneously dispersed throughout the glycerin in a container. Although not shown in the FIGURE, polymers B and C were also homogeneously dispersed throughout the glycerin. In contrast, even after the carboxyvinyl polymer was continuously stirred for 1 hour, murky white matter was partially suspended in the transparent glycerin. More specifically, the carboxyvinyl polymer was clumpy, and could not be homogeneously dispersed in glycerin.

### INDUSTRIAL APPLICABILITY

The gelled composition of the present invention has a range of applications, for example, in cosmetic materials, such as skin-care cosmetics, body-care cosmetics, and fragrance cosmetics. In particular, the high polyhydric alcohol content enables effective use of heat from mixing water with the polyhydric alcohol. Thus, the gelled composition is suitable for use in cosmetic materials, such as warm gels, and can also have applications in massage gels, sheet masks, cosmetic scrubs, and nourishing cosmetic materials.

1. A gelled composition comprising the following components (A) to (C):
(A) a polymer of an ethylenically unsaturated carboxylic acid monomer crosslinked with a water-soluble crosslinking agent;
(B) a polyhydric alcohol; and
(C) water,
wherein component (A) is present in an amount of 0.1 to 5 mass %, component (B) is present in an amount of 50 to 95 mass %, and component (C) is present in an amount of 1 to 10 mass %, based on the total amount of the gelled composition.

2. The gelled composition according to claim 1, wherein the polymer has a mean particle size of 5 to 40 μm.

3. The gelled composition according to claim 1, wherein the water-soluble crosslinking agent contains at least one compound selected from the group consisting of N,N′-methylenebis acrylamide, ethylene glycol dimethacrylate, ethylene glycol diglycidyl ether, polyethylene glycol dimethacrylate, polyethylene glycol diglycidyl ether, and sucrose allyl ether.

4. The gelled composition according to claim 1, wherein the water-soluble crosslinking agent contains sucrose allyl ether, and the degree of etherification of the sucrose allyl ether is 2.0 to 3.5.

5. A cosmetic material comprising the gelled composition according to claim 1.

6. The cosmetic material according to claim 5, which is a warm gel cosmetic material.
